(19) United States
(12) Patent Application Publication
(10) Pub. No.: US 2019/0008663 A1
(43) Pub. Date: Jan. 10, 2019

(54) PROSTHESIS SOCKET VACUUM DEVICE

(71) Applicant: KEN DALL ENTERPRISE CO., LTD., New Taipei City (TW)

(72) Inventors: Chia-Pao CHENG, New Taipei City (TW); Fu-Kuo WU, New Taipei City (TW); Hsiang-Ming Wu, New Taipei City (TW)

(21) Appl. No.: 15/641,338

(22) Filed: Jul. 5, 2017

(51) Int. Cl. A61F 2/80

(52) U.S. Cl. CPC A61F 2/80; A61F 2002/5073; A61F 2002/805

(57) ABSTRACT

A prosthesis socket vacuum device includes a base formed with a first chamber formed with a discharge channel and a vacuum relief channel and a second chamber provided, on an underside thereof, with a locking assembly, which includes a position-constraining block. A positioning assembly having sealing gaskets fit thereto includes a locking pin extending through a positioning member and formed with a recess. In setting the positioning assembly into the base, the positioning member compresses and expels internal air of the first chamber to the outside through the discharge channel in a soundless manner to form a vacuum condition. The locking pin drives the locking assembly to displace until the recess and a closure hole are fixed relative to each other so as to have the locking assembly closes the vacuum relief channel. A gap is provided between the closure hole and the recess to provide an effect of cushioning.

### (a) TECHNICAL FIELD OF THE INVENTION

The present invention relates generally to a prosthesis socket vacuum device, which includes a base and a positioning assembly that are connectable to each other in a more stable and quiet manner through an arrangement of a plurality of channels and sealing gaskets and provide an effect of cushioning and shock absorption after the connection.

### (b) DESCRIPTION OF THE PRIOR ART

To mount a limb prosthesis to a prosthesis user with a known structure, it is common to first wrap an inner lining (96) made of a soft material and a sealing strip (98) is fit outside the lining at a suitable location to work, in combination with a socket (95), to prevent undesired skidding thereby achieving the purpose of sealing.

Referring to FIG. 9, a socket base (90) is formed with a vertical through hole (91) extending through an interior thereof, and a toothed wheel (92) is arranged adjacent to an inside wall of the vertical through hole (91). The toothed wheel (92) is provided therein with a one-way bearing (93) for rotation in one given direction. The socket base (90) is coupled, with an outer side thereof, to a socket (95). The socket base (90) is also connected to a vacuum device (94). When a user puts on an inner lining (96) that is provided with a sealing strip (98), an arrestor axle (97) provided on a bottom of the inner lining (96) is used in combination with the vertical through hole (91), so that the toothed wheel (92) gets in engagement, in a position constrained manner, with a plurality of annular grooves (91) formed in the vertical through hole (91). When the arrestor axle (97) completely penetrates through the socket base (90), the vacuum device (94) is operated to evacuate air from an unsealed space (99) between the inner lining (96) and the socket (95) and inside the socket base (90) to set the interior of the entire structure in a vacuum condition to achieve the purpose of putting on a limb prosthesis.

The above-described process of putting on a limb prosthesis has numerous disadvantages, such as:

(1) To allow for complete extension through the socket base (90), the arrestor axle (97) is often made to have an extended length so that it often need to repeat the process of putting on once undesired positional shift occurs during the process of putting on; and if such a situation has not been noticed by the user, it may cause an unsmooth operation of the device during subsequent use of stepping or simply results in discomfort. The socket base (90) does not involve a well designed positioning measure so that it takes much time for the user to properly put the device on.

(2) The toothed wheel (92) has a one-way design for positioning purpose and in a regular process of inserting the arrestor axle (97), rotation is often caused during the process and gives off sound that might become annoying to other people staying around in a quite environment.

(3) The vacuum device (94) must be constantly actuated to evacuate residual air out of the unsealed space (99) in order to maintain stable mounting of the prosthesis, and thus, the prior art structure requires additional inclusion of the vacuum device (94). This results in additional weight and makes the structure complicated. In view of the prior art exhibiting such situations of being complicated and inconvenient operation in use thereof, there are still drawbacks that require further improvement.

### SUMMARY OF THE INVENTION

The present invention provides a prosthesis socket vacuum device, of which a general technical purpose is to provide an improvement on coupling between a base and a positioning assembly such that the coupling may easily achieve a vacuum condition and maintain stable connection by means of a plurality of sealing gaskets and channels and also to alleviate the sounding caused in making coupling with a prior art device by switching to a soundless operation (much more quite as compared to the prior art), but without the need for additional inclusion of extra device for air evacuation, while providing a certain degree of cushioning after coupling with the positioning assembly.

To achieve the above objective, the present invention provides a prosthesis socket vacuum device, which structurally comprises a base, which is formed, in an interior thereof, with a first chamber and a second chamber. The first chamber is formed therein with a discharge channel and a vacuum relief channel. The second chamber is provided, on an underside thereof, with a locking assembly. A positioning assembly is made up of a locking pin that extends through the positioning member. The locking pin has a lower portion formed with a recess and a plurality of second sealing gaskets are fit to one side of the recess. The positioning member comprises a plurality of first sealing gaskets fit to an outside surface thereof. When the positioning assembly is set into the base for coupling, the positioning member squeezes and expels air contained in the first chamber to the outside and also makes the locking pin drive the locking assembly to displace until the recess and the closure hole are fixed relative to each other, making the locking assembly close the vacuum relief channel. With a gap present between the closure hole and the recess, a certain degree of buffering may be provided after the positioning assembly is coupled to the base.

The present invention is made such that the positioning assembly is provided with a plurality of sealing gaskets for coupling with the base that is provided with the discharge channel and the vacuum relief channel so as to effectively form a vacuum condition through expelling air to the outside through the channels, allowing a mounting operation performed by a user not to make any noise and also forming a mechanism of buffering to enhance comfortableness of wearing.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of the present invention.

FIG. 2 is a perspective view of the present invention.

FIG. 3 is a cross-sectional view taken along line A-A of FIG. 2 showing a positioning assembly being set into a base.

FIG. 4 is a cross-sectional view taken along line A-A of FIG. 2 showing the positioning assembly is set and positioned in the base.

FIG. 5 is a cross-sectional view taken along line B-B of FIG. 2 showing air being discharged to the outside through a discharge channel.

FIG. 6 is a cross-sectional view taken along line B-B of FIG. 2 showing buffering achieved with the positioning assembly.

FIG. 7 is a cross-sectional view taken along line B-B of FIG. 2 showing a locking assembly being pushed to release vacuum.

FIG. 8 is an enlarged view of a part of the present invention shown in FIG. 6.

FIG. 9 is a schematic view illustrating a prior art structure.

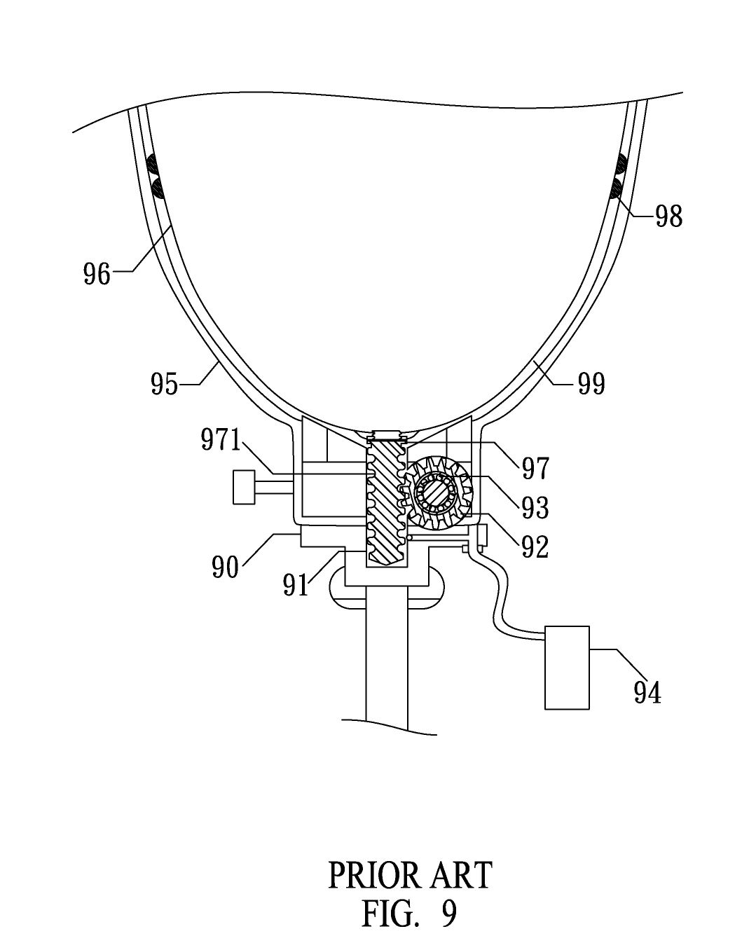

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

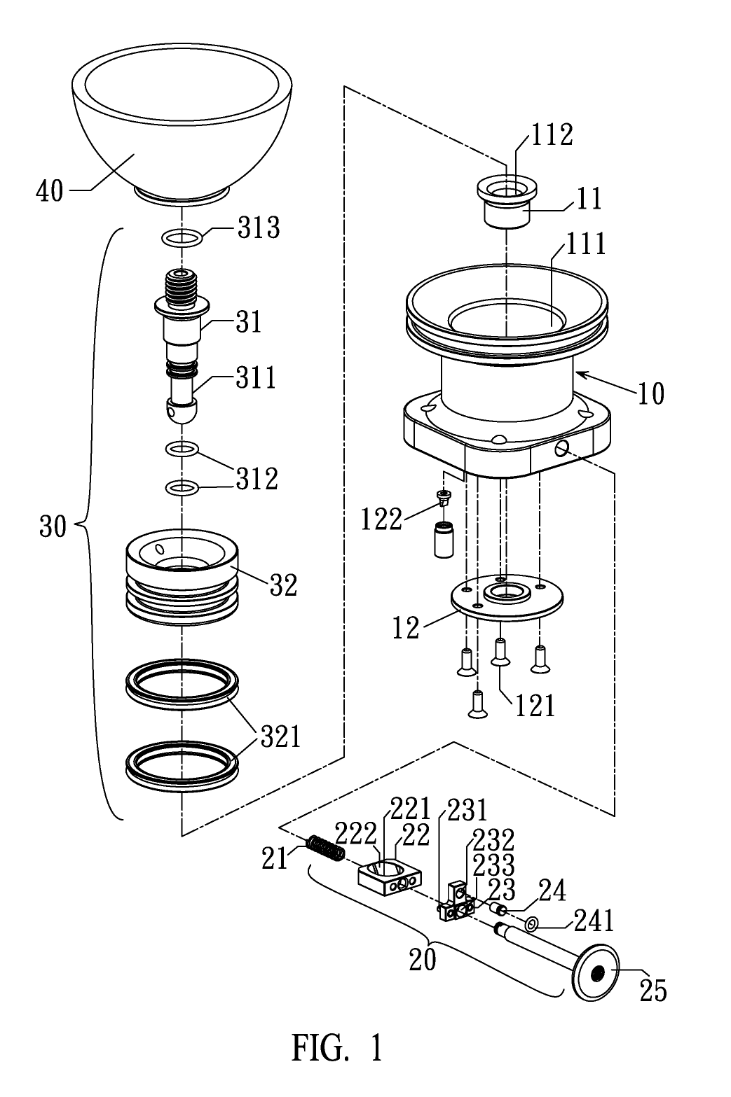

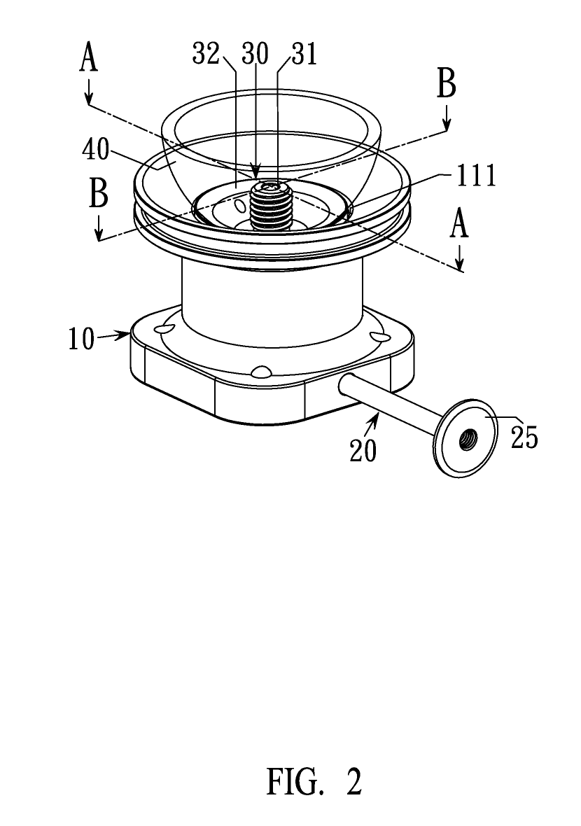

Better understanding of the present invention can be made through a detailed description given below generally for a preferred feasible embodiment according to the present invention with reference to FIGS. 1 and 2. The present invention relates to a prosthesis socket vacuum device, and more particularly to a structure for coupling between a socket prosthesis-lining (40) and a limb prosthesis, of which a general structure comprises a base (10), wherein the base (10) is formed with a first chamber (111) and a second chamber (112) arranged longitudinally in a center thereof. The first chamber (111) is formed therein with a discharge channel (13) extending to and in communication with a bottom of the base (10) and a vacuum relief channel (14), wherein the discharge channel (13) is provided therein with a one-way valve (122).

A locking assembly (20) comprises, assembled in sequence, a position-constraining block (22), a connection member (23), and a push member (25). The connection member (23) is of an inverted T-shape and the connection member (23) is formed with a first through hole (232) and a second through hole (233) respectively in upper and lower portions thereof. The first through hole (232) receive a valve block (24) to which a fourth seal gasket (241) is fit to insert and retain therein. The second through hole (233) receives the push member (25) to insert and retain therein. A plurality of projections (231) is arranged on a side surface of the second through hole (233) for connection with the position-constraining block (22). The locking assembly (20), as being set in a biased and pushed condition by a spring (21), is disposed under the second chamber (112), and a bottom lid (12), in combination with a plurality of fixing bolts (121), is threadingly fixed to the base (10) to retain the locking assembly (20) in an interior of the base (10).

A positioning assembly (30) has an upper portion configured for the socket prosthesis-lining (40) that is fit to a stump of a user. The positioning assembly (30) is formed of a locking pin (31) extending through a positioning member (32). The locking pin (31) has an upper portion that is provided with a third sealing gasket (313) at a site of coupling to the lining to prevent entry of air. The locking pin (31) has a lower portion that is provided with a recess (311) and is provided, on a side surface adjacent to the recess (311), with a plurality of second sealing gaskets (312) fit thereto. The positioning member (32) has an outside surface over which a plurality of first sealing gaskets (321) is fit. When the positioning assembly (30) is set into the base (10), the positioning member (32) squeezes and expels air contained in the first chamber (111) to move through the discharge channel (13) to discharge, in a soundless manner, to the outside by way of the one-way valve (122). When the positioning member (32) is squeezing the first chamber (111), air that is located outside the inner lining (40) and inside the socket (50) is not expelled into the first chamber (111), and only the air that is originally contained in the first chamber (111) will be discharged to the outside so as to generate a vacuum condition inside the first chamber (111) and also allow the locking pin (31) to drive the locking assembly (20) to move until the recess (311) and a closure hole (221) are fixed with respect to each other so that the locking assembly (20) closes the vacuum relief channel (14). The sealing gaskets described above can be adjusted in respect of the quantity thereof in view of actual needs for applications and are not limited to what described herein.

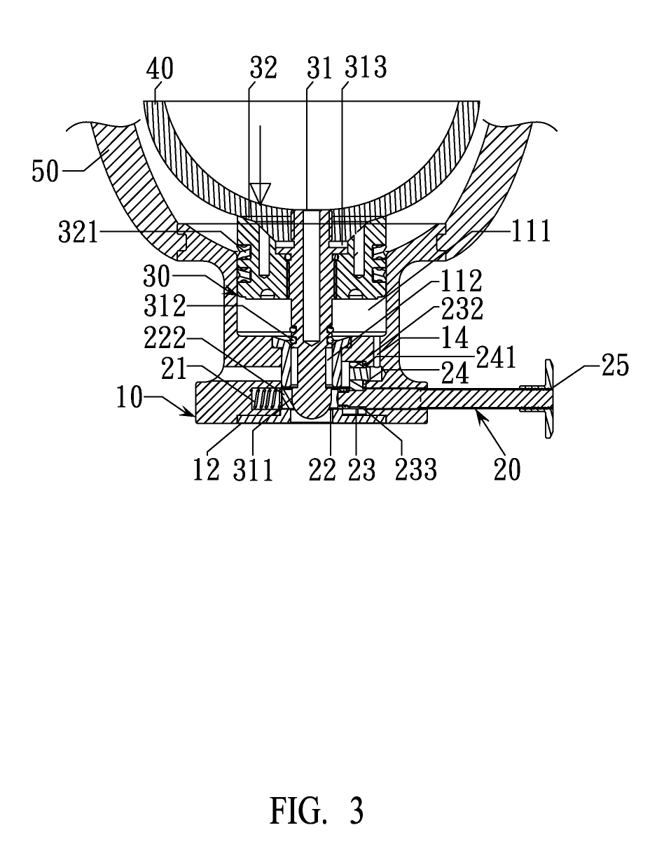

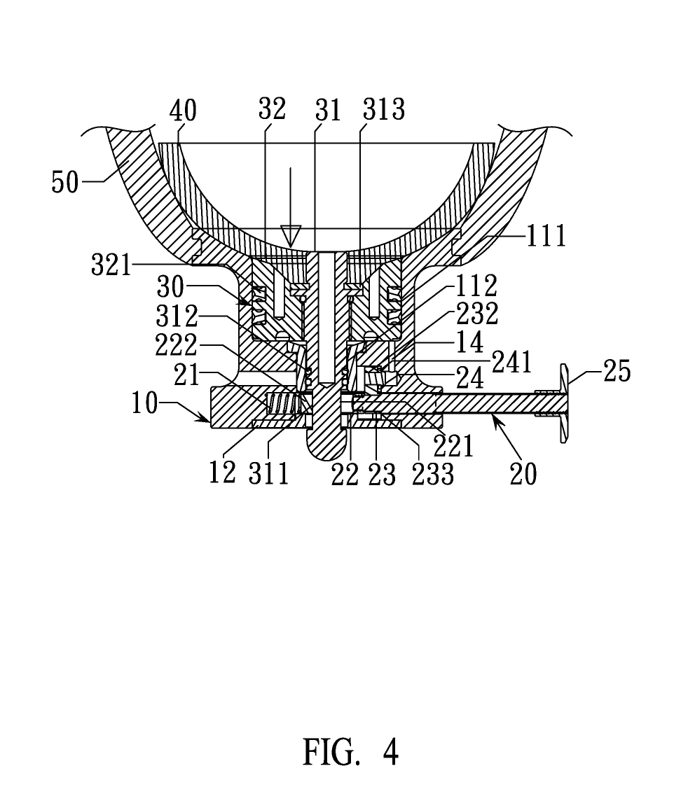

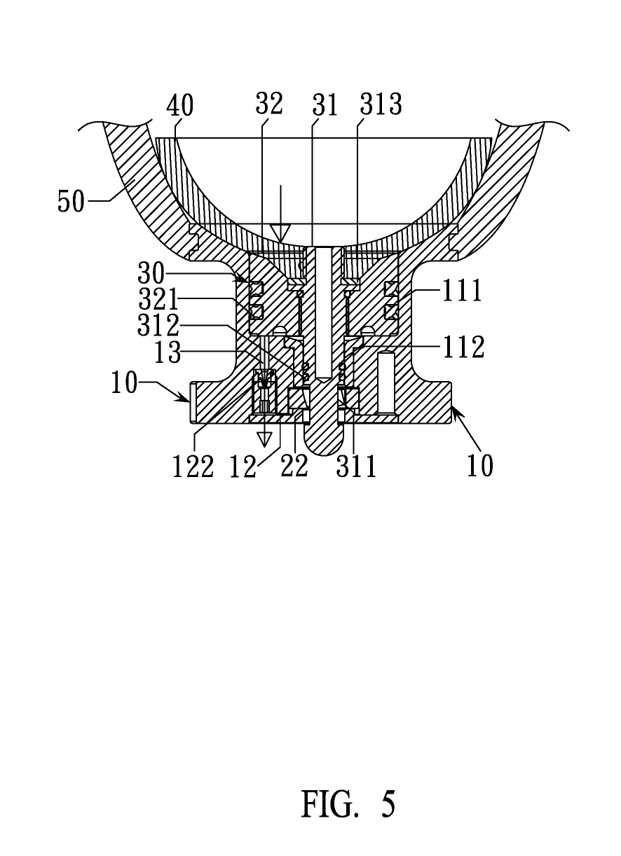

Referring to FIGS. 3-5, when the positioning assembly (30) is set into the interior of the base (10), the lower portion of the locking pin (31) is moved through the closure hole (221) of the position-constraining block (22). Due to an inclination surface (222) arranged inside the closure hole (221), the positioning assembly (30), when moved from a top side of the base (10) toward a bottom side, is allowed to slide along the inclination surface (222) so that the locking pin (31) can easily move, in one given direction, the locking assembly (20); and when the locking pin (31) further moves to a bottommost position, the recess (311) and the closure hole (221) are fixed in position with respect to each other, as being in alignment with each other, so that the locking assembly (20) is caused by a spring force of the spring (21) for position restoration thereby making the positioning assembly (30) completely coupled to and retained in the interior of the base (10).

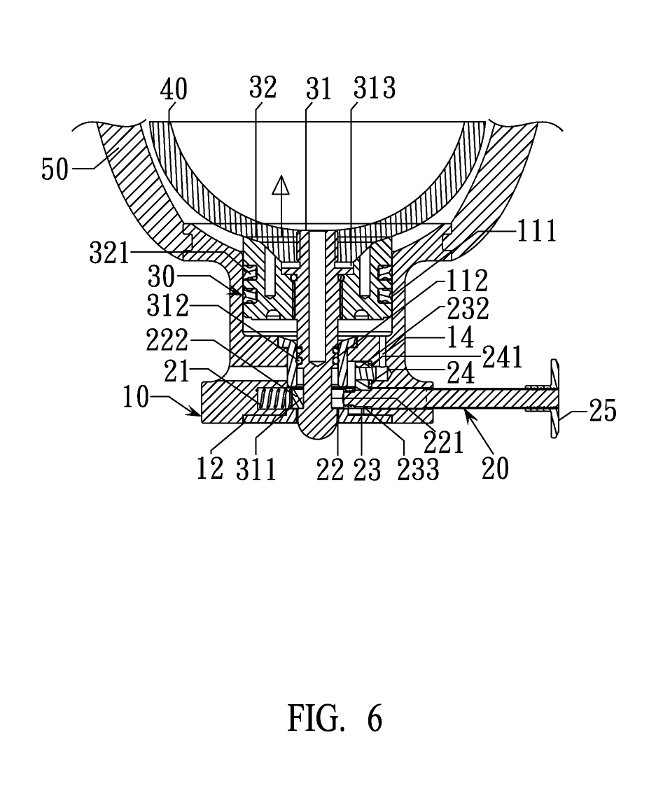

Referring to FIG. 6, with the positioning assembly (30) coupled to and retained in the interior of the base (10), when a user make a step, a force applied downward for treading causes a reaction force from the ground so as to cause the positioning assembly (30) to undergo repeated up and down displacement. The recess (311) and the closure hole (221), although kept retained to each other, have a gap therebetween to cushion the reaction force so caused. Further, the vacuum relief channel (14) is in engagement with and closed by the valve block (24) so that external air is not allowed to flow into the base (10).

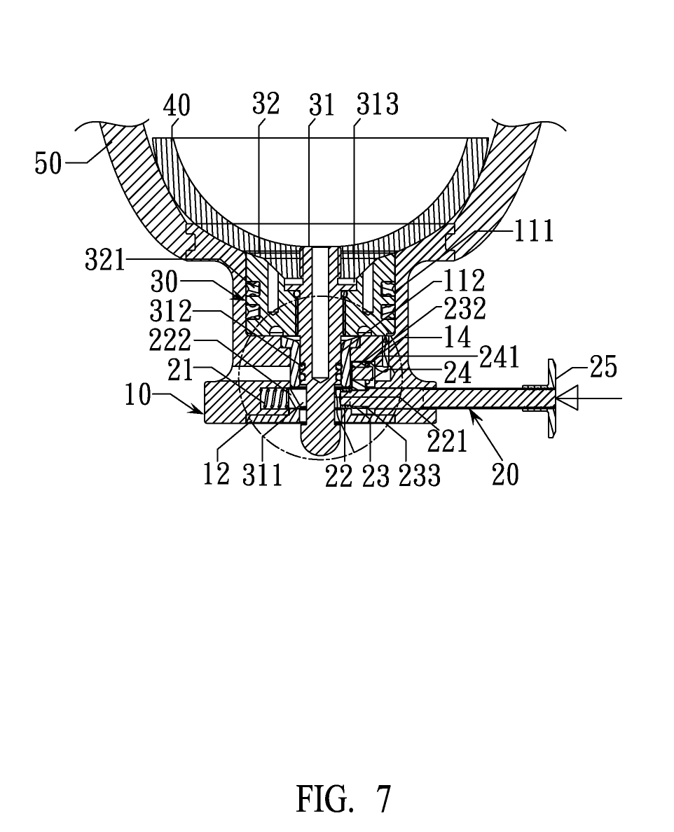

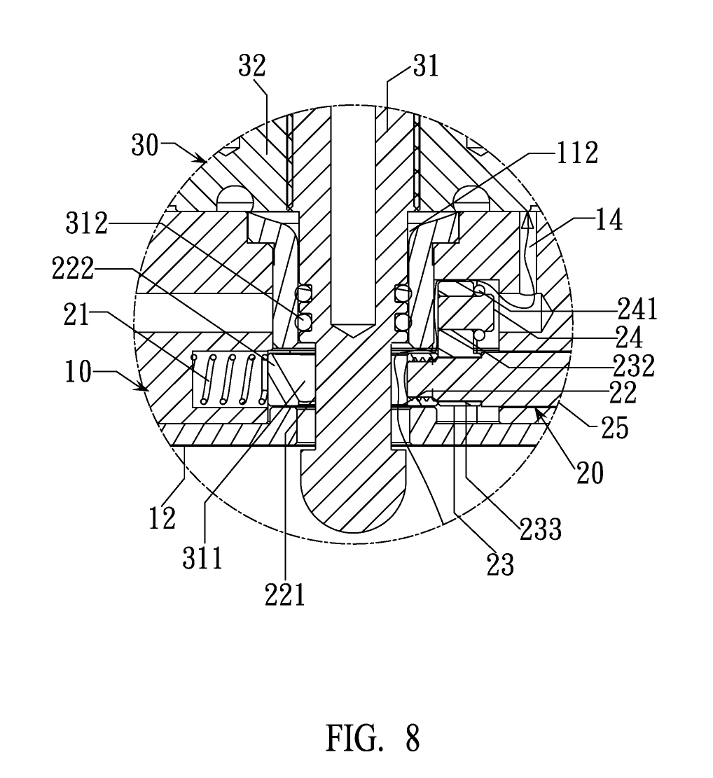

Referring to FIGS. 7-8, to remove the positioning assembly (30) from the interior of the base (10), the locking assembly (20) must be pushed and moved so as to make the valve block (24) disengage from and move away from the vacuum relief channel (14), allowing external air to flow in through the bottom of the base (10), and the position-constraining block (22) releases the recess (311) to allow easy removal of the positioning assembly (30).

The present invention provides a prosthesis socket vacuum device, which comprises a locking pin (31) that is reduced in size and used in combination with sealing gaskets so that a user may uses a length-reduced or shorter positioning assembly (30) to couple with the base (10) thereby improving the issue of poor coupling. Further, during coupling, the base (10) discharges internal air through the discharge channel (13), and the vacuum relief channel (14) to prevent flowing of air so as to achieve the purpose of completely fixing without additional inclusion of devices for air evacuation and vacuuming thereby reducing the load carried by the user. Further, the positioning assembly (30) and the locking assembly (20) provide certain spacing or gap therebetween to help buffer or cushion reaction force induced by stepping of the user so as to make the user feel comfortable.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A prosthesis socket vacuum device, comprising: a base, which is formed with a first chamber and a second chamber in a longitudinal direction, the first chamber being provided with a discharge channel extending to and in communication with a bottom of the base, and a vacuum relief channel; a locking assembly, which has an end provided with a position-constraining block, which is set, as being biased by a spring, under the second chamber, and an opposite end extending through the base to the outside, the position-constraining block being formed, in an interior thereof, with a closure hole; a positioning assembly, which comprises a locking pin extending through a positioning member, the locking pin having a lower portion formed with a recess, with a plurality of second sealing gaskets fit thereto at one side of the recess, the positioning member comprising a plurality of first sealing gaskets fit to an outside surface thereof, wherein when the positioning assembly is set into the base, the positioning member squeezes and expels air contained in the first chamber to discharge, in a soundless manner, to the outside through the discharge channel, and meanwhile, the locking pin drives the locking assembly t displace until the recess and the closure hole are fixed relative to each other to allow the locking assembly to close the vacuum relief channel, and wherein a gap is present between the closure hole and the recess to provide an effect of cushioning after the positioning assembly is coupled to the base.

2. The prosthesis socket vacuum device according to claim 1, wherein the positioning assembly further comprises a connection member, which comprises a plurality of projections for connection with the position-constraining block and is formed with a first through hole and a second through hole, the first through hole receiving a valve bock that comprise a fourth seal gasket fit thereto to insert and retain therein, the second through hole receiving a push member to insert and retain therein.

3. The prosthesis socket vacuum device according to claim 2, wherein pressing down the positioning assembly for a second time allows the position-constraining block to move away from the vacuum relief channel, so as to allow air to flow into the first chamber to release vacuum.

4. The prosthesis socket vacuum device according to claim 1, wherein the discharge channel further comprises a one-way valve arranged in an interior of the discharge channel to allow the positioning member to carry out expelling air without reversal of flow.
